(19) United States
(12) Patent Application Publication
(10) Pub. No.: US 2024/0358970 A1
(43) Pub. Date: Oct. 31, 2024

(54) FLUID DELIVERY SYSTEMS, DEVICES, AND RELATED METHODS

(71) Applicants: Boston Scientific Scimed, Inc., Maple Grove, MN (US); Boston Scientific Medical Device Limited, Galway (IE)

(72) Inventors: Deepak Kumar SHARMA, Muzaffarnagar (IN); Sharath Kumar G, Kanakapura (IN); Kevin L. BAGLEY, Natick, MA (US); Mazen YATIM, Southborough, MA (US); Samuel GOSHEN, Brookline, MA (US)

(73) Assignees: Boston Scientific Scimed, Inc., Maple Grove, MN (US); Boston Scientific Medical Device Limited, Galway (IE)

(21) Appl. No.: 18/645,594

(22) Filed: Apr. 25, 2024

Related U.S. Application Data
(60) Provisional application No. 63/498,353, filed on Apr. 26, 2023.

(51) Int. Cl. A61M 25/00

(52) U.S. Cl. CPC A61M 25/0082; A61M 25/0075; A61M 25/0097; A61M 2210/1042

(57) ABSTRACT

A medical device may comprise: a handle including an actuator, an end container configured to be coupled to a distal end of another medical device, the end container including a cavity including a movable portion, at least one channel, and a distal opening; a control element coupling the actuator to the movable portion of the end container. A proximal movement of the actuator may move the movable portion of the end container proximally within the cavity of the end container. The cavity may be configured to hold a fluid, and the proximal movement of the movable portion is configured to apply a compressive force to the fluid so that the fluid is expelled distally through the distal opening.

### CROSS-REFERENCE TO RELATED APPLICATIONS

This application claims the benefit of priority under 35 U.S.C. § 119 from U.S. Provisional Application No. 63/498,353, filed Apr. 26, 2023, which is incorporated by reference herein in its entirety.

### TECHNICAL FIELD

The disclosure relates generally to systems, devices, and methods for delivering fluid. More specifically, aspects of the disclosure pertain to systems, devices, and/or methods for delivering viscous fluid to a target site, via medical devices, such as endoscopes.

### BACKGROUND

Bleeding ulcers, for example, in a subject's gastrointestinal (GI) tract, may be treated via various treatments, including injection therapies. Injection therapies may require the delivery of a fluid, oftentimes, a highly viscous fluid, to the wound site(s) to form a protective layer that minimizes delayed bleeds, potential perforations, stricture formations, etc. Fluid channels of devices configured for fluid delivery may be of a small diameter, and highly viscous fluids may require a large amount of force to flow through narrow openings and fluid channels. Common fluid delivery devices, e.g., syringes, generally deliver fluid when a push force, i.e., a distal force, is applied to a feature of such devices, and a push force may be smaller than desirable.

### SUMMARY

This disclosure includes fluid delivery systems, devices, and methods of use thereof, e.g., methods of delivering fluid to a target site of a subject, for example, to help heal an ulcer and/or to perform hemostasis. Each of the aspects disclosed herein may include one or more of the features described in connection with any of the other disclosed aspects.

In an example, a medical device may comprise: a handle including an actuator, an end container configured to be coupled to a distal end of another medical device, the end container including a cavity including a movable portion, at least one channel, and a distal opening; a control element coupling the actuator to the movable portion of the end container. A proximal movement of the actuator may move the movable portion of the end container proximally within the cavity of the end container. The cavity may be configured to hold a fluid, and the proximal movement of the movable portion is configured to apply a compressive force to the fluid so that the fluid is expelled distally through the distal opening.

Any of the examples described herein may include any of the following features. The end container may further include a cap sealing a proximal end of the end container. The cap may include an opening through which the control element extends. The cap further may include a valve in fluid communication with the cavity. The at least one channel may be adjacent against an inner surface of the end container. The at least one channel may extend from a distal end of the end container to a proximal point that is distal from a proximal end of the end container, thereby allowing for a passage of fluid between the proximal end of the end container and a proximal opening of the at least one channel. The cavity of the end container may be filled with a viscous fluid. The proximal movement of the movable portion may flow the viscous fluid proximally towards a proximal opening of the at least one channel and distally through the at least one channel towards the distal opening. The cavity may be annular and at the least one channel includes a plurality of channels, and each of the plurality of channels is equidistant from one another in the annular cavity. The at least one channel may include a plurality of channels, and the end container may include a plurality of distal openings along a distal surface of the end container. The handle may include a handle body. The actuator may be movable in a first direction and a second direction relative to the handle body to control the movement of the one or more control elements. The movable portion may be a plunger configured to apply the compressive force to a fluid within the cavity. The plunger may include a first part and a second part stacked upon the first part. The first part may comprise a different material than the second part. The plunger may include at least one O-ring. The end container may be annular and includes a central opening. The end container may include at least one lighting element.

In another aspect, a medical device may comprise a handle including an actuator and an end container. The end container may be annular and include a central passage configured to receive another medical device. The end container may further include a cavity, a movable portion, at least one channel, and at least one opening along a distal surface of the end container. The medical device may further comprise one or more control elements coupling the actuator to the movable portion of the end container. A proximal movement of the actuator may move the movable portion of the end container proximally within the cavity of the end container. The at least one channel may include a proximal opening for receiving fluid from the cavity.

Any of the examples described herein may have any of the following features. The movable portion may be a plunger configured to apply compressive force to a fluid within the cavity so that the fluid flows proximally towards the proximal opening of the at least one channel and distally through the at least one channel and towards the at least one opening. The end container may further include a cap sealing a proximal end of the end container. The one or more control elements may extend through the cap. The cap further may further include a valve in fluid communication with the cavity.

In a further example, a medical device may comprise: a handle including an actuator and an end container. The end container may include a first cavity including a first movable portion, a second cavity including a second movable portion, a partition between the first cavity and the second cavity, and a distal chamber in fluid communication with the first cavity and the second cavity. The medical device may further comprise one or more control elements coupling the actuator to the first movable portion of the end container and the second movable portion of the end container. A proximal movement of the actuator may move the first movable portion proximally within the first cavity and the second movable portion proximally within the second cavity. The first cavity may be configured to hold a first fluid and the second cavity may be configured to hold a second fluid. The proximal movement of the actuator may be configured to apply a compressive force to the first fluid and the second fluid so that the first fluid and the second fluid flow into the distal chamber.

Any of the examples described herein may include any of the following features. The first cavity may further include a first inner casing concentric with the end container. The first inner casing may define a first inner cavity in fluid communication with the first cavity. The first movable portion may be movable within the first inner cavity.

Any of the examples described herein may have any of these features in any combination.

It may be understood that both the foregoing general description and the following detailed description are exemplary and explanatory only and are not restrictive of the invention, as claimed. As used herein, the terms “comprises,” “comprising,” “having,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1A depicts a perspective view of an exemplary medical device.

FIG. 1B depicts a transparent view of a portion of the exemplary medical device of FIG. 1A.

FIG. 1C depicts a perspective view of a feature of the exemplary medical device of FIG. 1.

FIG. 2A depicts a perspective view of another exemplary medical device.

FIG. 2B depicts a transparent view of a portion of the exemplary medical device of FIG. 2A.

FIG. 2C depicts a transparent, cross-sectional view of a portion of the exemplary medical device of FIG. 2A.

FIGS. 2D-2E depicts cross-sectional views of a portion of the exemplary medical device of FIG. 2A.

FIGS. 3A and 3B depict perspective views of another exemplary medical device.

FIG. 4A depicts a perspective view of another exemplary medical device.

FIG. 4B depicts a transparent view of a portion of the exemplary medical device of FIG. 4A.

FIG. 4C depicts a perspective view of a portion of the exemplary medical device of FIG. 4A.

FIG. 5 depicts a perspective view of another exemplary medical device.

### DETAILED DESCRIPTION

Reference is now made in detail to examples of this disclosure, aspects of which are illustrated in the accompanying drawings. Wherever possible, the same reference numbers will be used throughout the drawings to refer to the same or like parts.

Embodiments of this disclosure seek to improve and ease a user's ability to deliver a highly viscous fluid via a device fitted over a medical scope or a device delivered through a working channel of a medical scope, help perform wound treatment and/or hemostasis within the subject, and reduce overall procedure time, among other aspects. Each of the embodiments of this disclosure is configured to deliver viscous fluid when a pull force, i.e., a proximal force, is applied to a respective feature. The presence of a pull feature for fluid delivery, as opposed to a push feature for fluid delivery, may allow for an easier application of force and a sufficient amount of force to deliver viscous fluid through the various openings and channels of the embodiments of this disclosure. Examples of fluid, e.g., biocompatible viscous fluids, include, but are not limited to, fibrin, fluids including calcium salts, cyanoacrylates, albumin and glutaraldehyde, poly (ethylene glycol) (PEG), polyurethane, and fibrin. Such fluids may be endoscopically delivered adhesives that create a protective layer that minimizes delayed bleeds, potential perforations, and stricture formations.

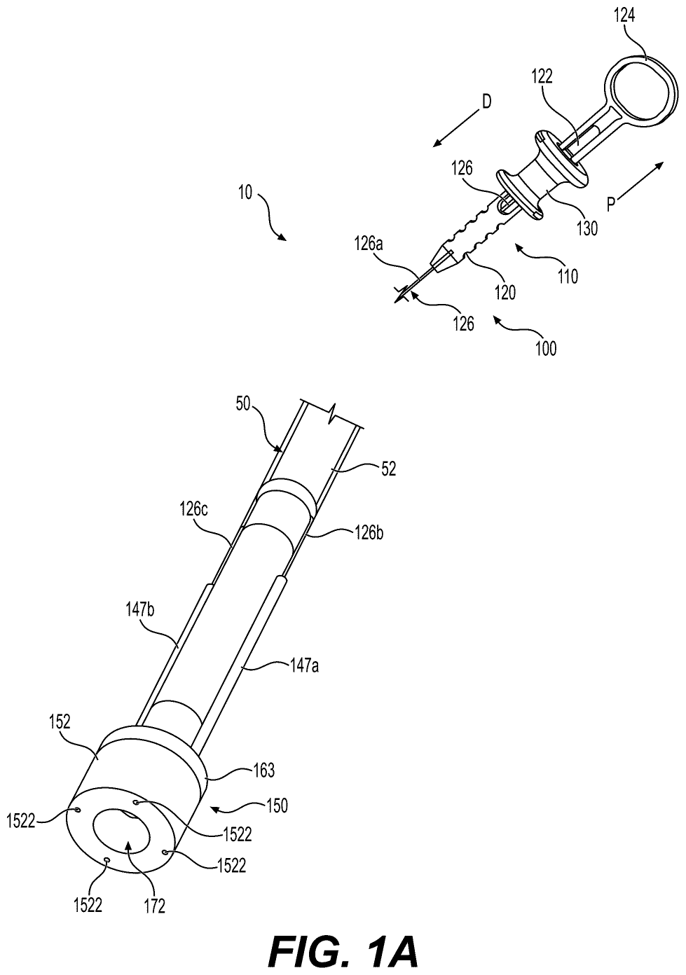

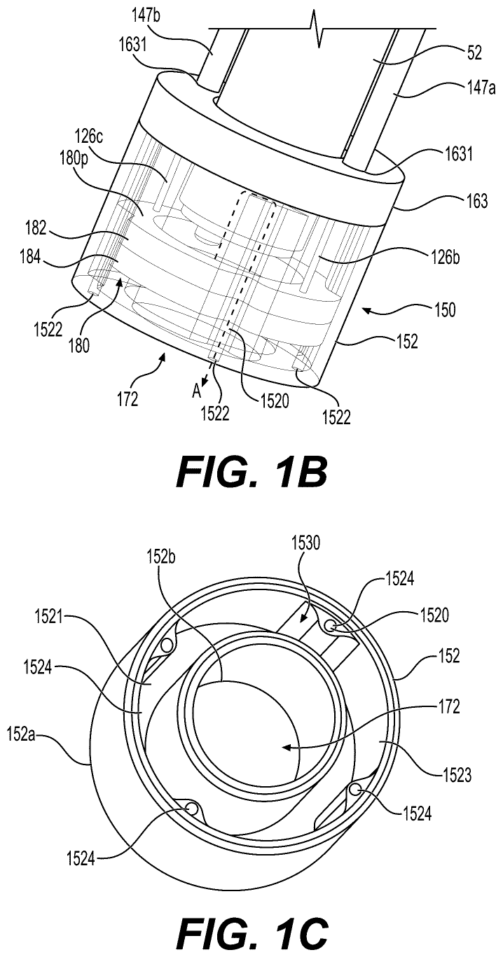

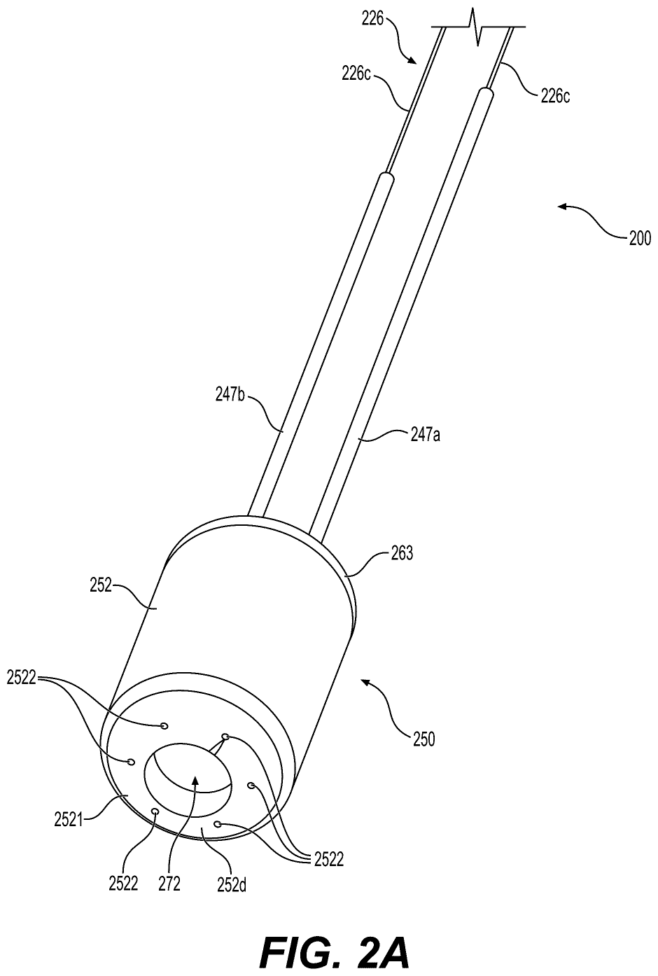

FIG. 1A-1C illustrate a medical device 100. As shown in FIGS. 1A and 1B, one or more portions of medical device 100 may be coupled to another or second medical device, for example, an endoscope 50, to form a medical system 10. Elements of medical system 100 and medical device 10 are not necessarily shown to scale.

Endoscope 50 may include a generally cylindrical tubular shaft 52, and may include a proximal portion (not shown) and a distal portion. Although not shown, the proximal portion may include or otherwise be coupled to a handle, for example, including one or more ports, controls, levers, electrical or communication connections, etc. Additionally, endoscope 50 may include one or more internal lumens or working channels, for example, extending longitudinally through shaft 52. In these aspects, the internal lumens or working channels may extend through the proximal portion and distal portion of shaft 52, for example, terminating distally at one or more distal openings (i.e., in a distal most end of endoscope 50).

Endoscope 50 (i.e., shaft 52 of endoscope 50) is not particularly limited. For example, shaft 52 may include a diameter of approximately 9 mm to approximately 15 mm, for example, approximately 10.5 mm to approximately 12 mm, or any size and/or shape configured to be coupled to an end container, e.g., a distal container 150 (discussed in further detail below), of medical device 100. As mentioned, endoscope 50 may include one or more internal lumens, for example, a working channel with a diameter of approximately 2 mm to approximately 4 mm, for example, approximately 2.8 mm. Additionally, a distal end face (not shown) of endoscope 50 may include one or more illumination device(s) (e.g., one or more LEDs, optical fibers, and/or other illuminators) and/or one or more visualization device(s) (e.g., one or more cameras, one or more image sensors, endoscopic viewing elements, optical assemblies including one or more image sensors and one or more lenses, etc.).

Although not shown, one or more portions of endoscope 50 (i.e., a distal portion of shaft 52) may be deflectable, for example, via one or more knobs or other controls on a proximal handle of endoscope 50. In these aspects, shaft 52 of endoscope 50 may be maneuvered while being delivered to the treatment site and/or positioned relative to the treatment site, for example, in a retroflex position, which may be used when the treatment site is in the subject's esophagus, stomach, duodenum, colon, or other portion of the GI tract.

Although the treatment site is discussed as being in the subject's GI tract, this disclosure is not so limited, as the treatment site may be any internal lumen or other tissue within the subject. Potential clinical uses of the embodiments of this disclosure and exemplary viscous fluids include post resection (e.g., endoscopic mucosal resection, endoscopic submucosal dissection, polypectomy), suture line reinforcement, fistula sealing, and post peroral endoscopic myotomy site closure. Additionally, although endoscopes are referenced herein, it will be appreciated that the disclosure encompasses any medical devices having a working channel extending from a proximal end to a distal end, such as ureteroscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters.

Medical device 100 includes a handle 110 and a distal container 150, and, as discussed below, features of handle 110 may be coupled to features of distal container 150 via at least one control element 126.

Handle 110 is not particularly limited, and manipulation of one or more portions of handle 110 may help to maneuver, position and/or reposition, and/or deliver viscous fluid over tissue at the treatment site. While handle 110 is shown as the handle of medical device 100, handle 110 (or a similar handle) may also function as the handles of medical device 200 and medical device 300, both of which are later discussed in further detail.

Handle 110 may include a handle body 120 and an actuator 130. Handle body 120 is not particularly limited, and may extend longitudinally. Handle body 120 includes a slot or track 122 extending along at least a portion of a length of handle body 120. Track 122 defines an internal passage that extends radially inwards from an outer surface of handle body 120, and said internal passage may be in communication with a lumen (not shown) of handle body 120 that may house control element 126. Track 122 may receive a riding portion (not shown) of actuator 130, for example, that slidably translates along or within track 122. Aspects of handle body 120 unencumbered by actuator 130 may be held within a hand of the user. Handle body 120 further includes an end portion 124, for example, at a proximal end of handle body 120. End portion 124 may define a finger hole through which a user may insert or rest one or more fingers (e.g., the user's thumb), thereby securing the user's grip on handle body 120.

Actuator 130 may be a spool-like actuator that surrounds/sheaths a portion of handle body 120, and is configured to slidably translate along or within handle body 120, via track 122, in a proximal or distal direction (as indicated by the directional arrows P and D shown in FIG. 1A.)

Although not shown, handle 110 may also include one or more springs or biasing elements, for example, within the internal lumen of handle body 120, to bias the movement of actuator 130. For example, the one or more springs or biasing elements may distally bias the movement of actuator 130. In these aspects, the user may proximally translate actuator 130 relative to handle body 120, but once the proximal force from the user is removed, the one or more biasing elements may urge actuator 130 distally. Such biasing may help minimize any inadvertent delivery of fluid due to incidental proximal force being applied to actuator 130.

Control element 126 is not particularly limited, and may be formed by a pull wire, a coil, a tube, a sheath, a rod, etc. Moreover, while device 100 may include only one control element 126, the number of control elements 126 extending between handle 110 and distal container 150 is not particularly limited. As shown in FIGS. 1A and 1B, a proximal portion of control element 126 may be a single strand 126a, whereas a distal portion of control element 126 may split to two separate strands 126b, 126c. In alternative embodiments, there may be a plurality of single strand control elements as opposed to a split strand embodiment. A proximal portion of control element (single strand 126a) may be coupled to actuator 130 of handle 110 and a distal portion of control element (strands 126b, 126c) may be coupled to a movable portion, e.g., a plunger 180, of distal container 150.

Actuator 130 may be coupled to strand 126a such that a proximal portion or end of control element 126 is held, for example, within a cavity or an internal passage of actuator 130. Actuator 130 may be coupled to stand 126, for example, via a friction fit, an adhesive, a press fit, a crimping, or any other appropriate coupling mechanism. In this aspect, movement (i.e., proximal or distal movement) of actuator 130 relative to handle body 120 may control the extension or retraction of control element 126 relative to sheath element(s) 147a, 147b (discussed in detail below), and thus control the extension or retraction of plunger 180 relative to distal container 150 (FIG. 1B).

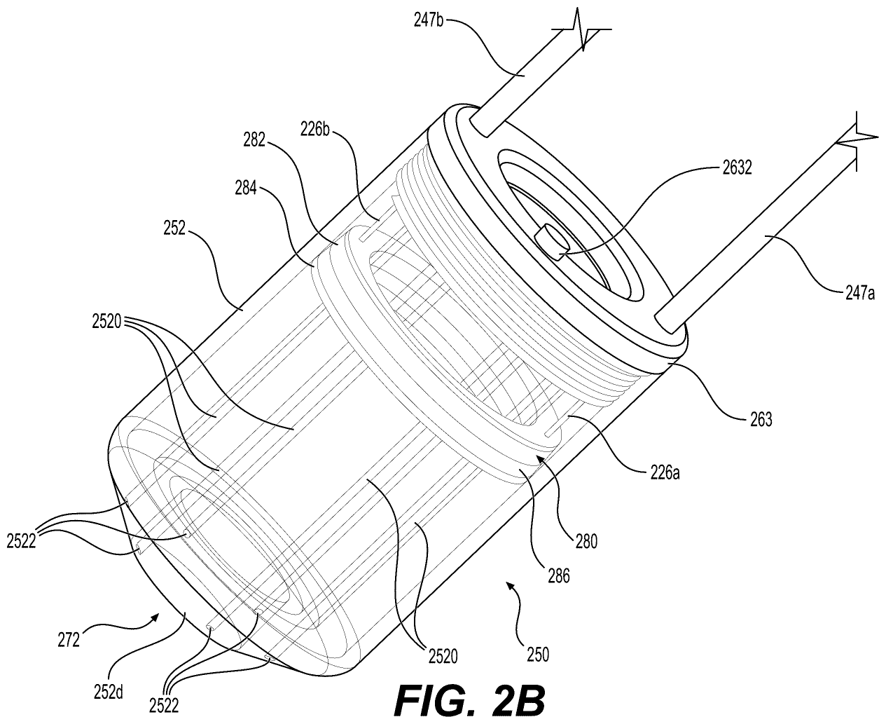

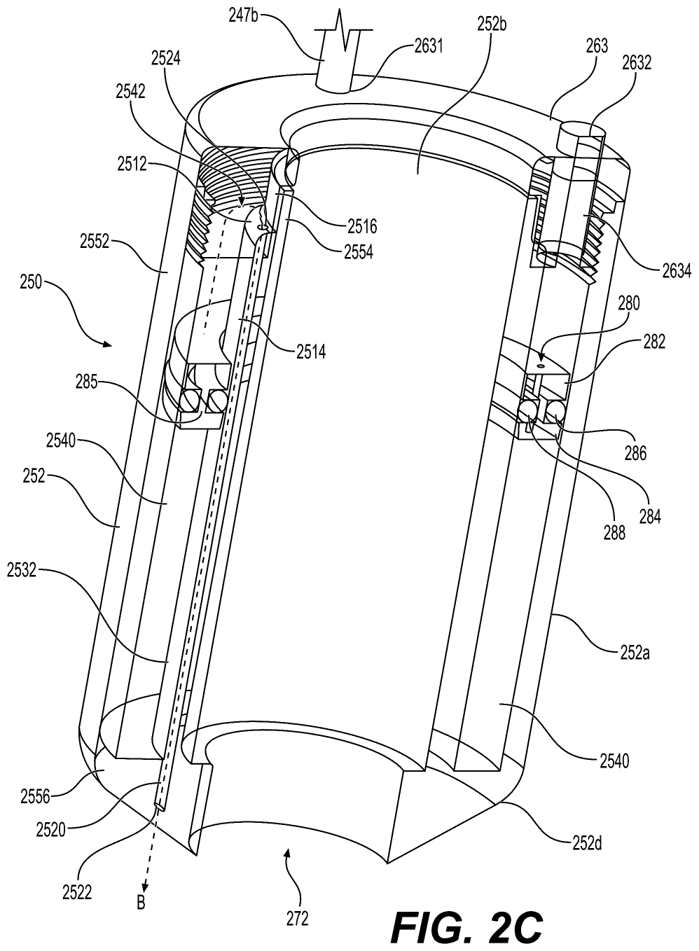

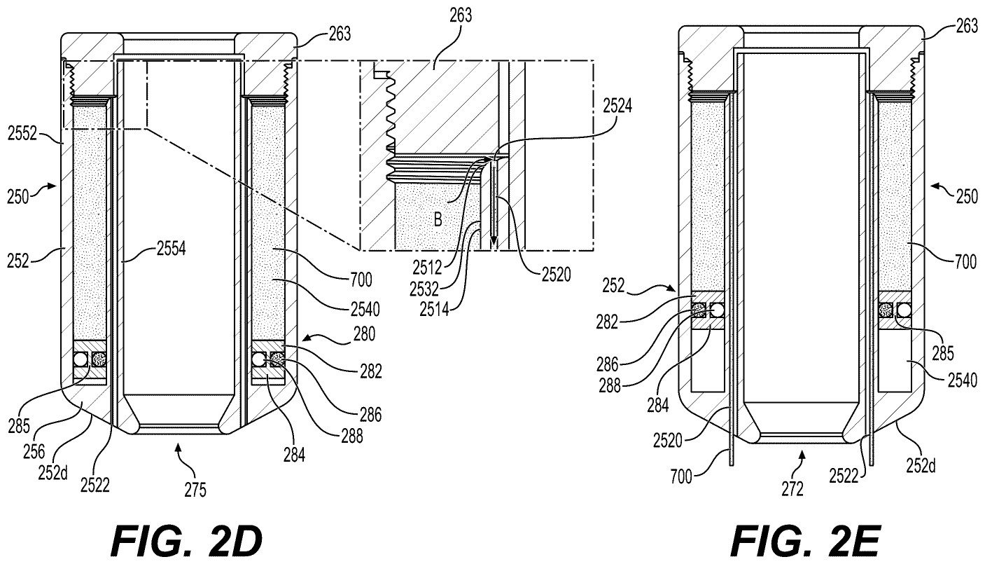

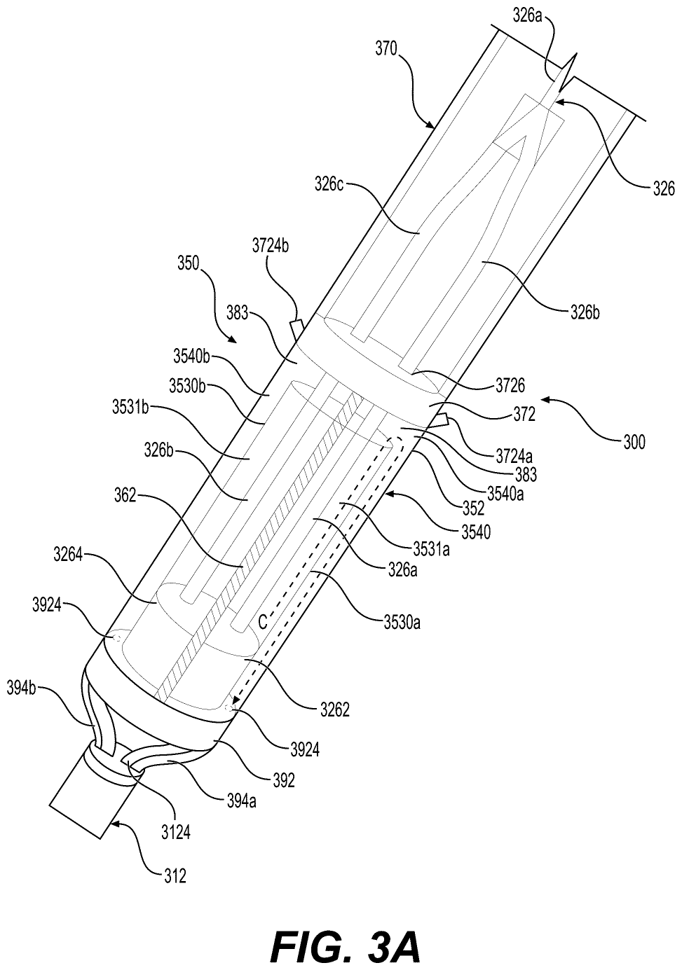

As shown in FIG. 1B, distal container 150 may include a proximal cap 163, a casing 152, and plunger 180. Proximal cap 163 is not particularly limited, and may be any suitable cap configured to be fitted within or over a proximal opening/end of casing 152, thereby sealing a chamber 1521 (FIG. 1C) within casing 152. In some embodiments, proximal cap 163, or a portion thereof, may comprise a flexible, frictious material, e.g., rubber, to securely engage with casing 152, thereby providing a seal to a proximal end of casing 152. Proximal cap 163 may further include a valve (not shown) which may be utilized to fill chamber 1521 with viscous fluid. Such a valve is not particularly limited, and may be any suitable valve that may provide passage between an opening of said valve and chamber 1521. For example, the valve may have any of the features of a valve 2632, discussed below with respect to FIGS. 2A-2C. Proximal cap 163 may further include a plurality of passages 1631 that extend from a proximal surface of cap 163 to a distal surface of cap 163. The shape and diameter of each of passages 1631 is not particularly limited. Each of passages 1631 may receive (or otherwise be coupled to or monolithically attached to) one of sheath elements 147a, 147b. Sheath elements 147a and 147b are not particularly limited, and may be any suitable sheath-like feature receiving one of strands 126b, 126c. Thus, each of strands 126b, 126c may pass through passages 1631, via sheath elements 147a, 147b, and towards plunger 180. The number of passages 1631 and sheath elements, e.g., 147a, 147b, is not particularly limited, and may correspond to the number of control element strands, e.g., 126b, 126c.

As shown in FIGS. 1B and 1C, casing 152 may be an annular or O-shaped cylindrical structure defining an outer circumference 152a and an inner circumference 152b. The open space within inner circumference 152b may define a central opening or passage 172. Passage 172 may be of any suitable shape and/or diameter that may receive a distal portion of shaft 52 of endoscope 50, so that shaft 52 may securely/snugly fit within passage 172. In some embodiments, a surface of casing 152 surrounding passage 172 may be lined or coated with (or otherwise include) frictious material to further secure the coupling between casing 152 and shaft 52 of endoscope 50.

As shown in FIG. 1C, casing 152 includes chamber 1521 and a plurality of channels 1520. Chamber 1521 is not particularly limited, and may be a space within the annular cylindrical structure of casing 152 that receives and holds viscous fluid. As previously noted, chamber 1521 may be filled with viscous fluid from a valve of proximal cap 163, or may be filled prior to being sealed by proximal cap 163.

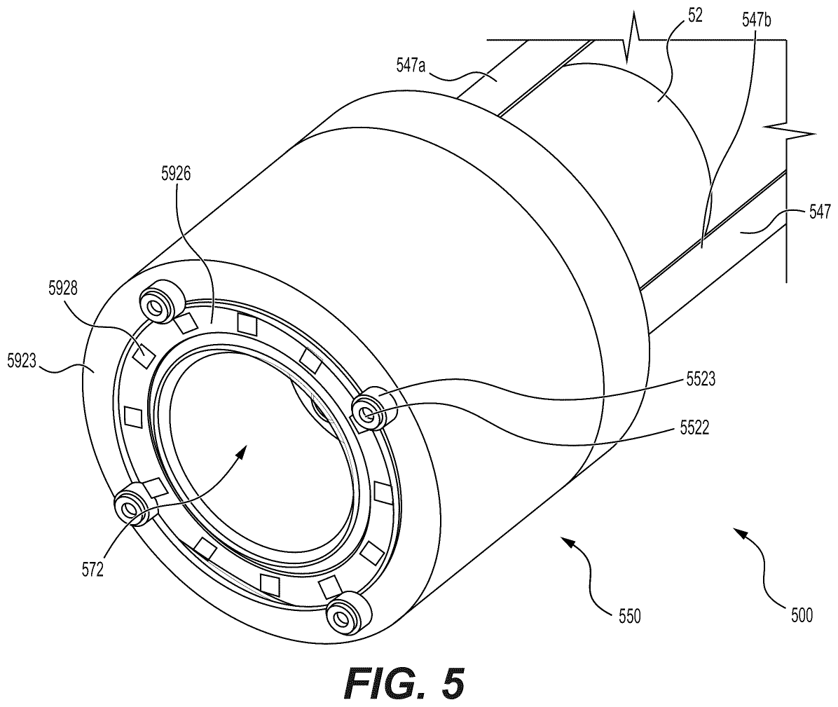

Casing 152 includes an inner surface 1523 of outer circumference 252athat partially surrounds chamber 1521 and extends from a distal end to a proximal end of casing 152. Inner surface 1523 may be lined with channel structures 1530 that extend between a distal end and a proximal end of inner surface 1523. Channel structures 1530 may protrude radially inward (toward inner circumference 152b) from inner surface 1523. For example, channel structures 1530 may extend from a distal end of inner surface 1523 to a proximal point of inner surface 1523 that may be distal to a proximal most end of inner surface 1523. In such an example, there may be a gap between a distal surface of proximal cap 163 and a proximal surface of channel structure 1530, thereby allowing passage of viscous fluid between the two surfaces. The number of channel structures 1530 is not particularly limited. Although four channel structures 1530 are depicted in FIG. 1C, that number is merely exemplary. As shown in FIG. 1C, each channel structure 1530 may be spaced apart equidistantly along a circumference of inner surface 1523 (e.g., at approximately 90-degree intervals for a configuration with four channel structures 1530), but is not necessarily limited to such spacing. Each of channel structures 1530 may define a distal opening 1522 (see FIGS. 1A-1B), a proximal opening 1524 (see FIG. 1C), and a channel 1520 extending therebetween. Thus, through such a configuration, viscous fluid may travel from chamber 1521 towards proximal opening 1524, through channel 1520, and be expelled from distal opening 1522, as shown by directional arrow A in FIG. 1B. Due to a viscosity of the fluid, the fluid generally will not flow out of distal opening 1522 until plunger 180 is actuated, as described in detail below. In addition or in the alternative, a valve, such as a valve 5523 (discussed below with respect to FIG. 5) may control or otherwise inhibit the fluid from flowing out of distal opening 1522.

As shown in FIG. 1B, a proximal surface 180p of plunger 180 is coupled to distal portions or ends of strands 126b, 126c of control element 126. Thus, as discussed above, the extension or retraction of control element 126 also translates plunger 180 within chamber 1521. Plunger 180 is not particularly limited, and may be any suitable feature configured to apply a compressive force to viscous fluid held within chamber 1521. To be able to efficiently apply such a force, plunger 180 may be of the same shape as chamber 1521 so that a contour of plunger 180 may be flush against inner surface 1523. For example, as shown in FIG. 1B, plunger 180 may have an approximately circular inner perimeter/circumference and an outer perimeter that is contoured to the shapes of channel structures 1530. For example, the outer perimeter may have segments that contour to inner surface 1523 (have an arcuate shape) and segments that are recessed and contour to a surface of channel structures 1530.

As shown in FIG. 1B, plunger 180 may include a first part 182 and a second part 184. First part 182 and second part 184 may be of the same shape and dimension, and may be stacked on top of one another via any suitable means (e.g., coupling, adhesion, etc.). In some examples, first part 182 and second part 184 may each have a washer-like shape (e.g., a washer-like shape with recesses along an outer perimeter thereof to accommodate channel structures 1530, as discussed above). First part 182 may be of a different material than second part 184. For example, first part 182 may be of an acrylic material, and second part 184 may be of a silicon or rubber-like material, such as, for example, nitrile, neoprene, ethylene propylene (EPDM rubber), silicone, fluorocarbon, and PTFE. Having two parts (i.e., first part 182 and second part 184) may allow for a greater volume of available space, as well as more surface area to prevent liquid from leaking.

Referring to FIGS. 1A-1C, an exemplary method of operating medical system 10 is further discussed. Distal container 150 may be pre-filled with fluid. Alternatively, a user may fill distal container 150 with viscous fluid via a valve on cap 163 prior to coupling medical device 100 to an insertion device. During operation, a user may first couple medical device 100 to an insertion device, such as endoscope 50, for example, by coupling distal container 150 to shaft 52 of endoscope 50, via inserting shaft 52 into passage 172. The user may extend actuator 130 to its distal most position (if not spring biased towards such a position). The user may then insert medical system 100 into the subject. The user may maneuver endoscope 50, using one or more visualization devices, illumination devices, etc. (not shown) at a distal tip of endoscope 50, to navigate a distal tip of shaft 52 to a treatment site within a body of a subject.

Once a distal portion of shaft 52 of endoscope 50 and end container 150 are positioned at the treatment site, the user may manipulate (e.g., proximally pull) actuator 130 relative to handle body 120 to translate plunger 180 proximally within chamber 1521 of distal container 150. Such manipulation of actuator 130 may proximally retract control element 126 to proximally translate plunger 180 against viscous fluid held within chamber 1521. Thus, plunger 180 may apply a compressive force to the viscous fluid so that the fluid may travel proximally, through the gap between channel structure 130 and proximal cap 163, to proximal openings 1524, through proximal openings 1524 of channel structures 1530, and distally through channels 1520, and be expelled distally towards the treatment site via distal openings 1522 (as shown by arrow A in FIG. 1B). In some aspects, endoscope 50 may include one or more visualization devices and/or one or more illumination devices (not shown), which may help the user visualize the treatment site and/or the position of distal container 150 relative to the treatment site.

FIGS. 2A-2E illustrate another exemplary embodiment, medical device 200. Similar to device 100, device 200 may also include a handle (e.g., handle 110), a control element 226, and a distal container 250. While a handle of device 200 is not shown, in FIGS. 2A-2E, handle 110 or a similar handle may function as the handle of device 200, as noted above. Said control element 226 of device 200 may also be similar to control element 126 of device 100, and may include a proximal portion of a single strand (similar to strand 126a) and a distal portion including strands 226b, 226c.

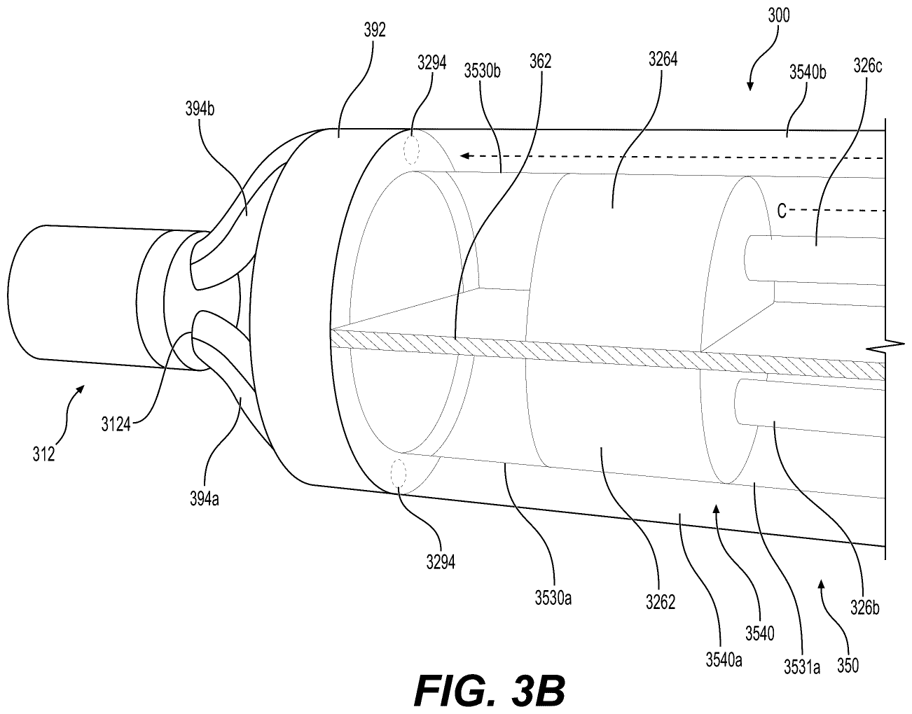

Like distal container 150, distal container 250 may include a proximal cap 263, a casing 252, and plunger 280. Proximal cap 263 may be similar (or identical) to proximal cap 163, and may seal a cavity 2521 of casing 252. As shown in FIG. 2B, proximal cap 263 may include a valve 2632 which may be utilized to fill cavity 2521 with viscous fluid. Valve 2632 may be any suitable valve providing a passage 2634 (FIG. 2C) between an opening of said valve and cavity 2521. Valve 2632 may selectively seal cavity 2521 when valve 2632 is not being used. For example, proximal cap 263 and a proximal portion of casing 252 may include threads that are used to screw proximal cap 263 on to casing 252. Proximal cap 263 may further include a plurality of passages 2631 (FIG. 3C) that extend from a proximal surface of cap 263 to a distal surface of cap 263. Like passages 1631, passages 2631 may receive (or otherwise be coupled to or monolithically attached to) one of sheath elements 247a, 247b, which, in turn, receive one of strands 226b, 226c. Thus, each of strands 226b, 226c may pass through passages 2631, via sheath elements 247a, 247b, and towards plunger 280.

Like casing 152, casing 252 may be an annular or O-shaped cylindrical structure defining an outer circumference 252a and an inner circumference 252b, as shown in FIG. 2C. The open space within inner circumference 252b may define passage 272. Passage 272 may be similar to passage 172, and may be of any suitable shape and/or diameter that may receive a distal portion of a shaft of an endoscope, so that an endoscope shaft may securely/snugly fit within passage 272. A distal surface 252d of casing 252 may vary from a distal surface of casing 152. As shown in FIGS. 2A-2C, a distal surface 252d of casing 252 may gradually protrude distally from outer circumference 252a of the distal surface towards inner circumference 252b of the distal surface, thereby defining a convex distal surface. A distal opening 2522 (described below) may be inclined towards inner circumference 252b, allowing the liquid to focus on a smaller (i.e., more concentrated area) area. The convex shape of the distal surface also may prevent any tissue trauma during navigation of device 200.

As shown in FIGS. 2B-2E, casing 252 includes a cavity 2540 and a plurality of channels 2520. Casing 252 may include an outer wall 2552, an inner wall 2554, and a distal wall 2556 extending between outer wall 2552 and inner wall 2554 at a distal end of casing 252. Outer wall 2552 and inner wall 2554 may be approximately concentric and may be approximately tubular shaped. Distal wall 2556 may extend approximately perpendicularly to outer wall 2552 and inner wall 2554.

Cavity 2540 may refer to the space or void within casing 252 which may receive and hold viscous fluid, e.g., fluid 700 (shown in FIGS. 2D and 2E). Cavity 2540 may be formed between a radially inner surface of outer wall 2552 and a radially outer surface of inner wall 2554. Cavity 2540 is not particularly limited and may also be annular or O-shaped so that cavity 2540 may be concentric with outer circumference 252a and inner circumference 252b of casing 252. Cavity 2540 may be accessible via valve 2632 of proximal cap 263, as passage 2634 may be in fluid communication with cavity 2540, or when a proximal end of casing 252 is without proximal cap 263. Cavity 2540 may be in fluid communication with an opening 2542 at a proximal end of casing 252 (i.e., a proximal end of cavity 2540 may be open). In examples, opening 2542 may be annularly shaped. A distal end of cavity 2540 may be closed, such that cavity 2540 does not extend fully through a distal end of casing 252. In other words, a wall of casing 252 may form a closed distal end of cavity 2540.

As shown in FIGS. 2C and 2D, casing 252 includes an inner surface 2532 (which may be a radially outer surface of inner wall 2554) that partially surrounds cavity 2540. Inner surface 2532 may extend from a distal end of cavity 2540 to a proximal edge 2512. Proximal edge 2512 may be distal to proximal opening 2542 of cavity 2540. Moreover, a proximal portion of inner surface 2532 may taper radially inwards towards passage 272, thereby defining proximal edge 2512. Proximal edge 2512 may include a plurality of proximal openings 2524, which define the proximal openings of channels 2520. Proximal edge 2512 may have an approximately shelf-like shape. Proximal edge 2512 may extend between a distal portion 2514 of inner surface 2532 and a proximal portion 2516 of inner surface 2516. Distal portion 2514 and proximal portion 2516 may extend approximately parallel to one another (e.g., parallel to a longitudinal axis of container 250). Proximal portion 2516 may be radially inward relative to distal portion 2513. Proximal edge 2512 may taper radially outward in a proximal direction. Proximal edge 2512 may be curved or may be straight and may be angled with respect to distal portion 2514 and proximal portion 2516.

Channels 2520 may extend distally from proximal openings 2524 within casing 252, and may extend between proximal edge 2512 and a distal surface of casing 252. Channels 2520 may extend through inner wall 2554. Moreover, channels 2520 may be between cavity 2540 and a surface of casing 252 that defines inner circumference 252b/passage 270. The number of channels 2520 is not particularly limited. As shown in FIG. 2B, each channel 2520 may be spaced apart equidistantly around inner circumference 252b, but is not necessarily limited to such spacing. Each of channels 2520 may define a distal opening 2522 on the distal surface of casing 252 and proximal opening 2524 (see FIGS. 2C and 2D) on proximal edge 2512 of inner surface 2532. Thus, channels 2520 may extend through distal wall 2556. Channels 2520 may be in fluid communication with cavity 2540 via proximal openings 2524 and with an outside area distal of container 250 via distal openings 2522. Thus, through such a configuration, viscous fluid 700 may travel from chamber 2521 towards proximal openings 2524, distally through channel 2520, and be expelled from distal openings 2522, as shown by directional arrow B in FIGS. 2C and 2D.

As shown in FIGS. 2B-2E, plunger 280 may be within cavity 2540, and may be proximally and distally translatable within cavity 2540. A proximal surface of plunger 280 is coupled to distal portions or ends of strands 226b, 226c of control element 226. Thus, as discussed above with respect to FIGS. 1A-1C, the extension or retraction of strands 226b, 226c of a control element translates plunger 280 within cavity 2540. Plunger 280 is not particularly limited, and may be any suitable feature configured to apply a compressive force to viscous fluid 700 held within cavity 2540. Like plunger 180, plunger 280 may be of the same shape as cavity 2540 so that a contour of plunger 280 may be flush against the surfaces defining cavity 2540, e.g., inner surface 2532 and a radially inner surface of outer wall 2552. In some examples, plunger 280 may have an annular (e.g., washer) shape. Plunger 280 may include a first part 282 and a second part 284. First part 282 and second part 284 may be of the same shape and dimension, and may be coupled to one another via a connective portion 285 (FIG. 2C). Connective portion 285 may be of a thinner profile than first part 282 and second part 284, thereby forming a radially inner space between connective portion 285, inner surface 2532, first part 282, and second part 284, a radially outer space may also be formed between connective portion 285, a surface 2534 of inner casing 231, first part 282, and second part 284. First part 182 may be of a same or different material than second part 184. Plunger 280 may further include an inner O-ring 288 residing within said inner space of plunger 280, and an outer O-ring 286 residing within said outer space of plunger 280. Both O-rings may help inhibit any leakage past plunger 280, as plunger 280 translate proximally within cavity 2540.

Device 200 may be operable in the same manner as discussed above with respect to medical system 10 and device 100. For example, during operation, a user may couple medical device 200 to an insertion device, such as endoscope 50 (shown in FIG. 1A), for example, by coupling distal container 250 to shaft 52 (shown in FIG. 1A) via inserting shaft 52 into passage 272. The user may then insert endoscope 50 and device 200 into the subject.

Once a distal portion of shaft 52 of endoscope 50 and distal container 250 are positioned at the treatment site, the user may manipulate (e.g., proximally pull) actuator of the handle of device 200 (e.g., handle 110) to translate plunger 280 proximally within cavity 2540 of distal container 250, as shown in FIGS. 2D and 2E. Such manipulation of the handle actuator may proximally retract control element 226 to proximally translate plunger 280 against viscous fluid 700 held within cavity 2540. Thus, plunger 280 may apply a compressive force to viscous fluid 700 so that fluid 700 may travel proximally, to proximal openings 2524, through proximal openings 2524 of channels 2520, and distally through channels 2520, and be expelled distally towards the treatment site via distal openings 2522, as shown in FIGS. 2D and 2E.

FIGS. 3A-3B illustrate another exemplary embodiment, medical device 300. Medical device 300 may include similar features as those discussed above with respect to medical devices 100, 200. However, unlike medical devices 100, 200, medical device 300 may be deliverable to a treatment site via a working channel of a medical scope, e.g., endoscope 50 (FIG. 1A), rather than being attached over an outer surface of endoscope 50. Furthermore, as discussed below, medical device 300 may be operable to deliver two fluids that are mixed together before delivery.

Similar to medical device 100, device 300 may also include a handle (e.g., handle 110), a control element 326, and a distal container 350. While a handle of device 300 is not shown, handle 110 (FIG. 1A) or a similar handle may function as the handle of device 300, as noted above. Control element 326 of device 300 may also be similar to control element 126 of device 100, and may include a proximal portion of a single strand 326a and a distal portion including strands 326b, 326c. As shown in FIG. 3A, device 300 may further include a housing 370 enclosing at least a portion of control element 326. Housing 370 is not particularly limited and may be any suitable structure that extends between distal container 350 and a handle of device 300. For example, housing 370 may include a flexible sheath. Housing 370 may prevent control element 326 from kinking and may provide a compression force when control element 326 is pulled proximally. Housing 370 may include, for example, a coil or metal hypotube or a braided catheter/sheath. Although other examples described herein may not depict a structure similar to housing 370, it will be appreciated that any of the examples described herein may include such a housing 370.

Distal container 350 may include a proximal cap 372, an outer casing 352, a distal cap 392, and a distal chamber 312. Proximal cap 372 may seal a proximal opening of outer casing 352. Proximal cap 372 may be coupled to outer casing 352 via, e.g., adhesive sealant, welding, UV curing, or threads. As shown in FIG. 3A, proximal cap 372 may include a valve 3724a which may be utilized to fill a cavity 3540a with viscous fluid (e.g., a first viscous fluid) and a valve 3724b which may be utilized to fill a cavity 3540b with viscous fluid (e.g., a second viscous fluid). Both valve 3724a, 3724b may be any suitable valve providing a passage between an opening of the valves and the corresponding cavities. For example, valves 3724a, 3724b may have the properties of valve 2632 of device 200. Proximal cap 372 may further include a plurality of passages 3726 that extend from a proximal surface of cap 372 to a distal surface of cap 372, each of which receive one of strands 326b, 326c. Thus, each of strands 326b, 326c may pass through a respective passage 3726, and towards a respective plunger, i.e., a first plunger 3262 and a second plunger 3264, as discussed in detail below.

Outer casing 352 may be a cylindrical structure a defining cavity 3540. Outer casing 352 may include a partition 362 extending between a proximal end and a distal end of casing 352, thereby defining two separate cavities 3540a, 3540b. Partition 362 is not particularly limited and may be configured to seal each of cavities 3540a, 3540b from one another. Partition 362 may be positioned along a diameter of outer casing 352 so that cavities 3540a, 3540b may be of equal volume, e.g., 1.0 ml-5.0 ml. However, partition 362 is not necessarily limited to such positioning and may be positioned to define cavities of different volumes. Each of cavities 3540a, 3540b may be configured to receive and hold viscous fluid, and in some instance, different viscous fluids. For example, cavity 3540a may be configured to hold a fluid having a first set of properties or composition, e.g., fibrinogen, and cavity 3540b may be configured to hold a fluid having a second set of properties or composition, e.g., thrombin. As discussed in further detail below, the different viscous fluids may be mixed within distal chamber 312, thereby crosslinking the two viscous fluids to form the delivery fluid, e.g., fibrin, cyanoacrylates, albumin and glutaraldehyde, poly (ethylene glycol) (PEG), or polyurethane. Although not explicitly discussed for each example herein, it will be appreciated that any of the devices disclosed herein may deliver any of the fluids above.

Cavity 3540a may include a first inner casing 3530a. First inner casing 3530a may be a half cylindrical casing extending from a surface of partition 362 so that outer casing 352 and first inner casing 3530a may be concentric with one another. However, in some other embodiments, first inner casing 3530a may be cylindrical and partition 362 may include slots therein for receiving casing 3530a. First inner casing 3530a may longitudinally extend from a distal end of partition 362 to a proximal portion of partition 362, but not to a proximalmost end of partition 362, thereby defining a gap 383 between a proximal end of first inner casing 3530a and the proximal end of portion 362. Thus, a cavity 3531a defined by first inner casing 3530a and partition 362 may be in fluid communication with cavity 3540a. Likewise, cavity 3540b may include a second inner casing 3530b. Second inner casing 3530b may also be a half cylindrical casing extending from a surface of partition 362 so that outer casing 352 and second inner casing 3530b may be concentric with one another. Second inner casing 3530b may also longitudinally extend from a distal end of partition 362 to a proximal portion of partition 362, but not to the proximal most end of partition 362, thereby defining gap 383 between a proximal end of second inner casing 3530b and the proximal end of portion 362. Thus, a cavity 3531b defined by second inner casing 3530b and partition 362 may be in fluid communication with cavity 3540b.

As shown in FIGS. 3A-3B, a first plunger 3262 may be within the aforementioned cavity 3531a of inner casing 3530a, and may be proximally and distally translatable within inner casing 3530a. A proximal surface of first plunger 3262 is coupled to a distal portion or end of strand 326b of control element 326. Thus, the extension or retraction of strand 326b of control element 326 translates plunger 3262 within inner casing 3530a. First plunger 3262 is not particularly limited, and may be any suitable feature configured to apply a compressive force to viscous fluid held within cavity 3540a. First plunger 3262 may be of the same shape as cavity 3531a of inner casing 3530a so that a contour of plunger 3262 may be flush against the surfaces defining inner casing 3530a. Thus, as first plunger 3262 is translated proximally to apply a compressive force, viscous fluid held within cavity 3540a (including cavity 3531a) may travel in the direction as indicated by directional arrow C shown in FIG. 3A. Viscous fluid may move proximally through inner casing 3530a to the proximal opening of inner casing 3530a, and distally through outer casing 352 to a distal opening 3294 as discussed in further detail below.

Similarly, a second plunger 3264 may be within the aforementioned cavity 3531b of inner casing 3530b, and may be proximally and distally translatable within inner casing 3530b. A proximal surface of first plunger 3264 is coupled to a distal portion or end of strand 326c of control element 326. Thus, the extension or retraction of strand 326c of control element 326 translates plunger 3264 within inner casing 3530b. Second plunger 3264 is not particularly limited, and may be any suitable feature configured to apply a compressive force to viscous fluid held within cavity 3540b (including cavity 3531b). Second plunger 3264 may be of the same shape as cavity 3531b of inner casing 3530b so that a contour of plunger 3264 may be flush against the surfaces defining inner casing 3530b. Thus, as second plunger 3264 is translated proximally to apply a compressive force, viscous fluid held within cavity 3540b (including cavity 3531b) may travel in the direction as indicated by directional arrow C shown in FIG. 3A. Viscous fluid may move proximally through inner casing 3530b to the proximal opening of inner casing 3530b, and distally through outer casing 352 to a distal opening 3294 discussed in further detail below.

Given that both strands 326b, 326c are coupled to a single proximal strand 326a, and strand 326a is coupled to a single actuator (e.g., actuator 130 of handle 110), both plungers 3262, 3264 may necessarily translate simultaneously in either the proximal or distal directions. However, in other embodiments, each of strands 326b, 326c may extend towards a handle and be coupled to it respective actuator, so that each plunger 3262, 3264 may be translated independently from the other.

Distal cap 392 may seal a distal opening of outer casing 352. As shown in FIG. 3B, a proximal surface of distal cap 392 may abut against (or otherwise be coupled to, monolithically formed with, etc.) a proximal end of partition 362, via, e.g., adhesive sealant, UV curing, or threads. Moreover, a proximal surface of distal cap 392 may include openings 3294. The number of openings 3294 is not particularly limited. Distal cap 392 may be coupled to outer casing 352 so that at least one opening 3294 may be in fluid communication with cavity 3540a and at least one opening 3294 may be in fluid communication with cavity 3540b. Openings 3294 may be in fluid communication with passages (not shown) extending between a proximal surface and a distal surface of distal cap 392, and towards a passage 394a or 394b. For example, cavity 3540a may be in fluid communication with passage 394a via one opening 3294. Likewise, cavity 3540b may be in fluid communication with passage 394b via another opening 3294.

Passages 394a, 394b are not particularly limited, and each may be any tubular structure that may provide a passage for viscous fluid between casing 352/distal cap 392 and distal chamber 312. Distal openings (not shown) of passages 394a, 394b may be in fluid communication with distal chamber 312 via openings 3124 along a proximal surface of chamber 312. Thus, a first viscous fluid, e.g., fibrinogen (lyophilized pooled human concentrate), from cavity 3540a may flow into chamber 312 via passage 394a, and a second viscous fluid, e.g., thrombin (bovine origin, human thrombin, etc.), from cavity 3540b may flow into chamber 312 via passage 394b. This may allow for the two different viscous fluids to mix together in required proportions. As previously noted, the first viscous fluid may be different from the second viscous fluid, and both viscous fluids may crosslink with one another in chamber 312, thereby forming the delivery fluid, e.g., fibrin, that is to be applied to a treatment site.

Chamber 312 is not particularly limited and may be any suitable container configured to receive viscous fluid. A distal end of chamber 312 may include one or more outlets (not shown) configured to dispense the delivery fluid held within chamber 312. Said outlet(s) is not particularly limited.

Referring to FIGS. 3A-3B, an exemplary method of operating a medical system including medical device 300 is further discussed. A user may insert an insertion device, e.g., an endoscope, into the subject. The user may maneuver the endoscope, using one or more visualization devices, illumination devices, etc. (not shown) at a distal tip of the endoscope, to navigate a distal portion of the endoscope to a treatment site within a body of a subject. Once the distal portion of the endoscope is positioned at or adjacent to the treatment site, the user may then insert medical device 300 into a port, e.g., a Y-port, of the endoscope, for example, so that a distal portion of device 300 is within a working channel of the endoscope. The user may fill cavities 3540a, 3540b of distal container 350 with viscous fluids any time before inserting medical device 300 into the endoscope. As noted above, cavities 3540a, 3540b may be filled with the same or different viscous fluids.

The user may extend device 300 past a distal end of the endoscope and manipulate (e.g., proximally pull) actuator 130 (see FIG. 1A) relative to handle body 120 (see FIG. 1A) to translate plungers 3262, 3264 proximally within their respective inner casings 3530a, 3530b of distal container 350. For example, such manipulation of actuator 130 may proximally retract control element 326 to proximally translate plunger 3262 against the first viscous fluid held within cavity 3540a (including cavity 3531a) and plunger 3264 against the second viscous fluid held within cavity 3540b (including cavity 3531b). Thus, each of plungers 3262, 3264 may apply a compressive force to the first or second viscous fluid so that the fluid may travel through openings 3924 of distal cap 392, through passage 394a or 394b, and be expelled towards the treatment site via an outlet of distal chamber 312.

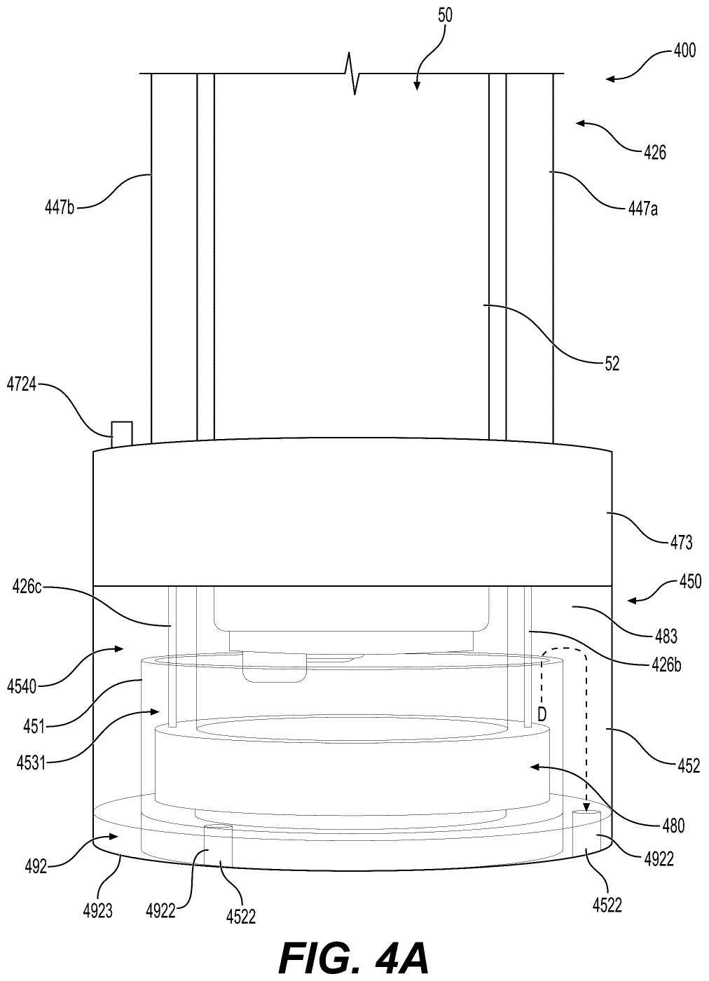

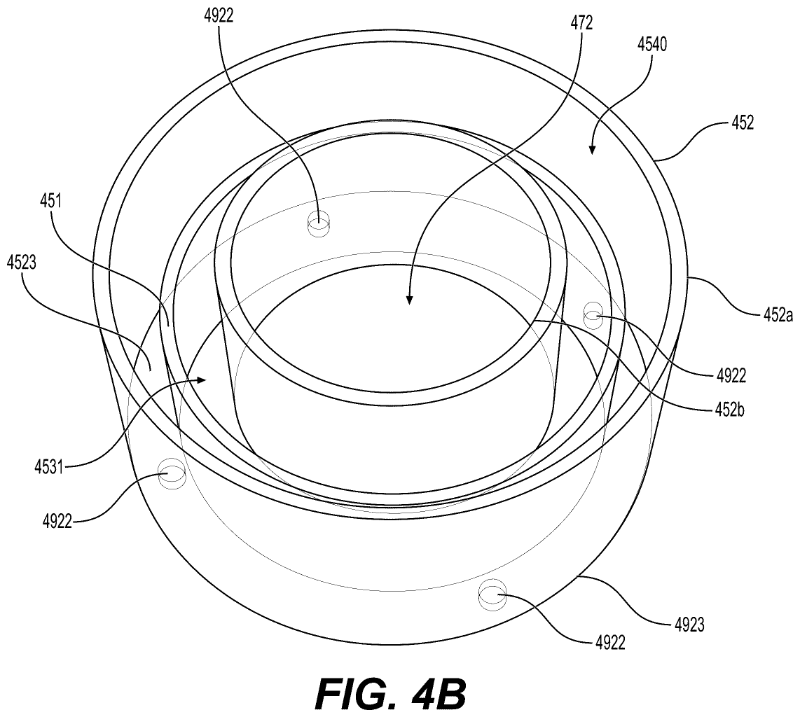

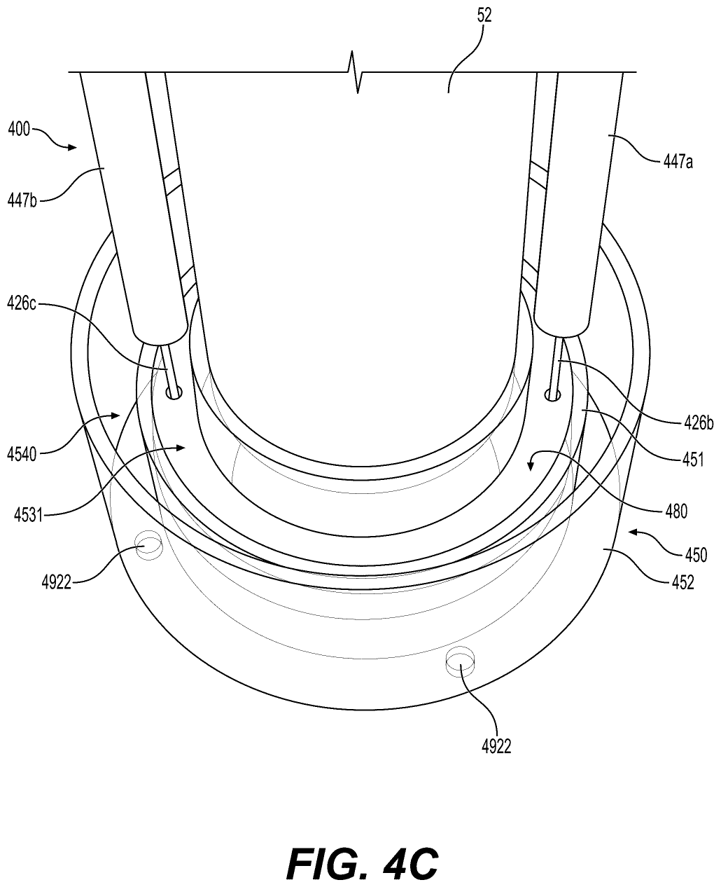

FIGS. 4A-4C illustrate another exemplary embodiment, medical device 400. Medical device 400 may include similar features as those discussed above with respect to medical device 300. However, unlike medical device 300, medical device 400 may be attached over an outer surface of endoscope 50 (FIG. 1A) and delivered to a treatment site.

Similar to medical device 300, device 400 may also include a handle (e.g., handle 110), a control element 426, and a distal container 450. While a handle of device 400 is not shown, handle 110 (FIG. 1A) or a similar handle may function as the handle of device 400, as noted above. Control element 426 of device 400 may also be similar to control element 126 of device 100, and may include a proximal portion of a single strand (similar to 126a) and a distal portion including strands 426b, 426c.

Distal container 450 may include a proximal cap 473 and an outer casing 452. Proximal cap 473 may seal a proximal opening of outer casing 452 via, e.g., adhesive sealant, UV curing, or threads. As shown in FIG. 4A, proximal cap 473 may include a valve 4724 which may be utilized to fill a cavity 4540 of casing 452 with viscous fluid. Valve 4724 may be any suitable valve providing a passage between an opening of valve 4724 and cavity 4540. For example, valve 4724 may have the properties of valve 2632 of device 200. Proximal cap 473 may further include a plurality of passages (not shown) that extend from a proximal surface of cap 473 to a distal surface of cap 473. Each of said passages may receive (or otherwise be coupled to or monolithically attached to) one of sheath elements 447a, 447b. Sheath elements 447a, 447b are not particularly limited, and may be any suitable sheath-like feature receiving one of strands 426b, 426c. Thus, each of strands 426b, 426c may pass through said passages of proximal cap 473, via sheath elements 447a, 447b, and towards plunger 480. The number of passages and sheath elements, e.g., 447a, 447b, is not particularly limited, and may correspond to the number of control element strands, e.g., 426b, 426c.

Like outer casings 152, 252, outer casing 452 may be an annular or O-shaped cylindrical structure defining an outer circumference 452a and an inner circumference 452b, as shown in FIG. 4B. The open space within inner circumference 452b may define passage 472. Passage 472 may be of any suitable shape and/or diameter that may receive a distal portion of a shaft of an endoscope, so that an endoscope shaft may securely/snugly fit within passage 472. Outer casing 452 may also include a distal wall 492. Distal wall 492 may define one or more passages 4922 extending between a proximal end of distal wall 492 and a distal end of distal wall 492. Each of one or more passages 4922 may include a distal opening 4522. Openings 4522 may be arranged along a distal surface 4923 of wall 492 in any suitable manner. For example, openings 4522 may be spaced apart equidistantly around distal surface 492, as shown in FIG. 4B. The number of distal openings 4522 is not particularly limited.

Outer casing 452 also defines a cavity 4540, which may receive and hold a viscous fluid, e.g., fibrin. As shown in FIG. 4A, casing 452 may be without a partition. However, in other embodiments, casing 452 may further include a partition, thereby defining two separate cavities, each of which is configured to receive the same or a different viscous fluid.

Cavity 4540 may include an inner casing 451. Inner casing 451 may be a secondary annular or O-shaped cylindrical structure housed within cavity 4540 so that inner casing 451 may be between outer circumference 452a and inner circumference 452b, and also concentric with the aforementioned circumferences. Inner casing 451 may proximally extend a certain distance from a distal inner surface 4523 of outer casing 452, but not up to a proximal most end of casing 452, thereby defining a gap 483 between a proximal end of inner casing 451 and the proximal end of outer casing 452. Thus, an inner cavity 4531 defined by inner casing 451 and inner circumference 452b of outer casing 452 may be in fluid communication with cavity 4540.

As shown in FIGS. 4A and 4C, a plunger 480 may be within the aforementioned inner cavity 4531, and may be proximally and distally translatable within inner casing 451. A proximal surface of plunger 480 is coupled to distal portions or ends of strands 426b, 426c of control element 426. Thus, the extension or retraction of strands 426b, 426c of control element 426 translates plunger 480 within inner casing 451. Plunger 480 is not particularly limited, and may be any suitable feature configured to apply a compressive force to viscous fluid held within cavity 4540 (and inner cavity 4531). Plunger 480 may be of the same shape as cavity 4531 of inner casing 451 (e.g., may have an annular/washer shape) so that a contour of plunger 480 may be flush against the surfaces defining inner casing 451. Thus, as plunger 480 is translated proximally to apply a compressive force, viscous fluid held within cavity 4540 (including cavity 4531) may travel in the direction as indicated by directional arrow D shown in FIG. 4A. Viscous fluid may move proximally through inner casing 451 to the proximal opening of inner casing 451, distally through outer casing 452 to distal openings 4522, and expelled distally from distal openings 4522.

Referring to FIGS. 4A-4C, an exemplary method of operating a medical system including medical device 400 is further discussed. For example, during operation, a user may couple medical device 400 to an insertion device, such as endoscope 50 (shown in FIG. 1A), for example, by coupling distal container 450 to shaft 52 (shown in FIG. 1A) via inserting shaft 52 into passage 472. The user may then insert endoscope 50 and device 400 into the subject.

Once a distal portion of shaft 52 of endoscope 50 and distal container 450 are positioned at the treatment site, the user may manipulate (e.g., proximally pull) actuator of the handle of device 400 (e.g., handle 110) to translate plunger 480 proximally within inner casing 451 of outer casing 452. Such manipulation of the handle actuator may proximally retract control element 426 to proximally translate plunger 480 against viscous fluid held within cavity 4540. Thus, plunger 480 may apply a compressive force to the viscous fluid so that the fluid may travel proximally to a proximal opening of inner casing 451, distally through outer casing 452, and be expelled distally towards the treatment site via distal openings 4522.

FIG. 5 depicts a further exemplary medical device 500. Medical device 500 may have any of the features of any of the medical devices described above. The features of medical device 500 may be incorporated into any of the other examples described herein. Aspects of medical device 500 are described herein so as to elucidate particular features of medical device 500. Discussion of certain aspects may be omitted here but may be the same as or similar to aspects of the medical devices described above. Medical device 500 may have a distal container 550, having any of the features of the distal containers described above. Medical device 500 may have one or more sheath elements 547, such as sheath elements 547a, 547b, as shown in FIG. 5. Sheath elements 547 may enclose control elements (not shown in FIG. 5), as described above. Medical device 500 may be coupled to a shaft 52 (e.g., of endoscope 50, shown and described above) of another medical device via a central opening or passage 572 of container 550, having any of the properties of the central openings or passages above.

Container 550 may have a distal surface 5923, having any of the properties (e.g., shape, size) of distal surface 4923 or any other distal surface of any of the devices described herein. Distal surface 5923 may include a plurality of distal openings 5522, which may have any properties of any distal openings described herein. Distal openings 5522 may be in communication with channels, casings, or other structures of container 550, such as any of the structures described above for holding and/or transmitting fluid. As shown in FIG. 5, distal surface 5923 may include four distal openings 5522; however, medical device 500 may include any suitable number of distal openings 5522. Distal openings 5522 may be incorporated into valves 5523. Any of the examples described above may incorporate valves having properties of valves 5523. Valves 5523 may help to prevent fluid from leaking out of distal openings 5522. Valves 5523 may be closed until a plunger or other actuator (such as any of the plungers/actuators described above) is activated to pressurize and/or deliver fluid. Pressure from the fluid on valves 5523 when the plunger or other actuator is activated may cause valves 5523 to at least partially open.

Distal surface 5923 also may incorporate lighting elements (e.g., ultraviolet (UV) or visible (VIS) light sources or sensors) that may be used to cure or otherwise activate the fluid delivered through distal openings 5522. For example, distal surface 5923 may include a substrate 5926. Substrate 5926 may have one or more lamps 5928 (e.g., filament lamps) or other light sources thereon. As shown in FIG. 5, substrate 5926 may have an annular shape (e.g., washer shape) and may be disposed about central opening or passage 572 and may have a plurality of lamps 5928 disposed thereon (e.g., twelve lamps 5928, as shown in FIG. 5, or any suitable number of lamps 5928). In alternatives, a plurality of substrates 5926 may be disposed on distal surface 5923, each having one or more lamps 5928 thereon. In an example, lamp 5928 may be an approximately 4 W UV lamp. In another example, lamp 5928 may be an approximately 150 W halogen (VIS) lamp. Although lamps 5928 are depicted as being substantially flat against distal surface 5923/substrate 5926, it will be appreciated that lamps 5926 may selectively or permanently extend distally and/or radially from distal surface 5923. For example, lamps 5926 may have gooseneck features to allow for focusing light in a desired direction. Lamps 5928 and/or sensors of distal surface 5923 may be coupled to conductive wires or other conductive elements that pass through sheath elements 547 or otherwise extend proximally.

In an exemplary method, one or more 4 W UV lamp(s) 5928 may have a 365 nm wavelength. Lamp(s) 5928 may be positioned directly over or otherwise adjacent to administered fluids (e.g., administered gels). Lamp(s) 5928 may be concentrated to apply light to an area of about 120 cubic centimeters. A maximum intensity of such lamp(s) 5928 may be approximately 33 mW/cm². In an alternative, one or more 150 W halogen lamp(s) 5928 may have gooseneck or other extendible features that focus the light over the administered fluid/gel. The light from lamp(s) 5928 may be concentrated on an area of approximately 8 π cubic centimeters. A maximum intensity of such lamp(s) 5928 may be approximately 6 W/cm². The parameters provided above are merely exemplary, and any suitable power levels, arrangements, or other features of lamps or other light sources may be utilized. In examples, a power value chosen for a lamp may depend upon a number of lamps utilized.

In addition, one or more light sensors (not shown) may be utilized to measure and/or adjust light emitted by lamp(s) 5928 or another light source (e.g., a light source separate from device 500). Such a light sensor may be positioned, for example, on distal surface 5923. Lamp(s) 5928 (or other light sources) may be automatically adjusted based on data from such sensors. In some examples, actuating a plunger or other actuator (such as any of the plungers/actuators described herein) may automatically deliver fluid and activate lamp(s) 5928. In other examples, lamp(s) 5928 may be activated by a separate control mechanism (e.g., button or switch).

While principles of this disclosure are described herein with the reference to illustrative examples for particular applications, it should be understood that the disclosure is not limited thereto. Although aspects of the disclosure are described with respect to the examples described herein, it will be appreciated that features of the various examples may be combined in any suitable manner. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a handle including an actuator;
an end container configured to be coupled to a distal end of another medical device, wherein the end container includes a cavity including a movable portion, at least one channel, and a distal opening; and
a control element coupling the actuator to the movable portion of the end container;
wherein a proximal movement of the actuator moves the movable portion of the end container proximally within the cavity of the end container, and
wherein the cavity is configured to hold a fluid, and the proximal movement of the movable portion is configured to apply a compressive force to the fluid so that the fluid is expelled distally through the distal opening.

2. The medical device of claim 1, wherein the end container further includes a cap sealing a proximal end of the end container, and wherein the cap includes an opening through which the control element extends.

3. The medical device of claim 2, wherein the cap further includes a valve in fluid communication with the cavity.

4. The medical device of claim 3, wherein the at least one channel is adjacent against an inner surface of the end container.

5. The medical device of claim 1, wherein the at least one channel extends from a distal end of the end container to a proximal point that is distal from a proximal end of the end container, thereby allowing for a passage of fluid between the proximal end of the end container and a proximal opening of the at least one channel.

6. The medical device of claim 1, wherein the cavity of the end container is filled with a viscous fluid.

7. The medical device of claim 6, wherein the proximal movement of the movable portion flows the viscous fluid proximally towards a proximal opening of the at least one channel and distally through the at least one channel towards the distal opening.

8. The medical device of claim 1, wherein the cavity is annular and the at least one channel includes a plurality of channels, and each of the plurality of channels is equidistant from one another in the annular cavity.

9. The medical device of claim 1, wherein the at least one channel includes a plurality of channels, and the end container includes a plurality of distal openings along a distal surface of the end container.

10. The medical device of claim 1, wherein the handle includes a handle body, and wherein the actuator is movable in a first direction and a second direction relative to the handle body to control the movement of the one or more control elements.

11. The medical device of claim 1, wherein the movable portion is a plunger configured to apply the compressive force to a fluid within the cavity.

12. The medical device of claim 11, wherein the plunger includes a first part and a second part stacked upon the first part, wherein the first part comprises a different material than the second part.

13. The medical device of claim 12, wherein the plunger includes at least one O-ring.

14. The medical device of claim 1, wherein the end container is annular and includes a central opening.

15. The medical device of claim 1, wherein the end container includes at least one lighting element.

16. A medical device, comprising:
a handle including an actuator;
an end container, wherein the end container is annular and includes a central passage configured to receive another medical device, wherein the end container further includes a cavity, a movable portion, at least one channel, and at least one opening along a distal surface of the end container;
one or more control elements coupling the actuator to the movable portion of the end container;
wherein a proximal movement of the actuator moves the movable portion of the end container proximally within the cavity of the end container, and
wherein the at least one channel includes a proximal opening for receiving fluid from the cavity.

17. The medical device of claim 16, wherein the movable portion is a plunger configured to apply compressive force to a fluid within the cavity so that the fluid flows proximally towards the proximal opening of the at least one channel and distally through the at least one channel and towards the at least one opening.

18. The medical device of claim 16, wherein the end container further includes a cap sealing a proximal end of the end container, wherein the one or more control elements extend through the cap, and wherein the cap further includes a valve in fluid communication with the cavity.

19. A medical device, comprising:
a handle including an actuator;
an end container, wherein the end container includes:
a first cavity including a first movable portion;
a second cavity including a second movable portion;
a partition between the first cavity and the second cavity;
and a distal chamber in fluid communication with the first cavity and the second cavity;
one or more control elements coupling the actuator to the first movable portion of the end container and the second movable portion of the end container;
wherein a proximal movement of the actuator moves the first movable portion proximally within the first cavity and the second movable portion proximally within the second cavity, and
wherein the first cavity is configured to hold a first fluid and the second cavity is configured to hold a second fluid, and the proximal movement of the actuator is configured to apply a compressive force to the first fluid and the second fluid so that the first fluid and the second fluid flow into the distal chamber.

20. The medical device of claim 19, wherein the first cavity further includes a first inner casing concentric with the end container, wherein the first inner casing defines a first inner cavity in fluid communication with the first cavity, and wherein the first movable portion is movable within the first inner cavity.
